(19) United States
(12) Patent Application Publication
(10) Pub. No.: US 2008/0312194 A1
(43) Pub. Date: Dec. 18, 2008

(54) Methods and compositions for normalizing meibomian gland secretions

(76) Inventors: George W. Ousler, III, North Andover, MA (US); Matthew J. Chapin, Amesbury, MA (US); Mark B. Abelson, Andover, MA (US); George Minno, Suwanee, GA (US); Aron Shapiro, Somerville, MA (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/074,028

(22) Filed: Feb. 28, 2008

Related U.S. Application Data
(60) Provisional application No. 60/904,209, filed on Feb. 28, 2007.

(51) Int. Cl. A61K 31/65; A61P 43/00

(52) U.S. Cl. 514/152

(57) ABSTRACT

The present invention provides non-aqueous compositions for normalizing meibomian gland secretions, and methods of use thereof. The present invention further provides compositions and methods for treating and/or preventing the signs and/or symptoms of dry eye disease.

### REFERENCE TO RELATED APPLICATIONS

This application claims the benefit of U.S. Provisional Application No. 60/904,209, filed Feb. 28, 2007, the contents of which are incorporated by reference in its entirety.

### FIELD OF THE INVENTION

The present invention relates generally to novel compositions and methods for normalizing meibomian gland secretion and the treatment and prevention of diseases related thereto. More specifically, the present invention relates to ophthalmic compositions comprising one or more active agents, e.g., an anti-infective agent and/or anti-inflammatory agent, and a non-aqueous component, useful for the normalization of abnormal meibomian gland secretions. The invention additionally relates to methods of administering such compositions to a subject in need thereof.

### BACKGROUND

Tears are comprised of three layers. The mucus layer coats the cornea forming a foundation so the tear film can adhere to the eye. The middle aqueous layer provides moisture and supplies oxygen and other important nutrients to the cornea. The outer lipid layers is an oily film that seals the tear film on the eye and helps to prevent evaporation of the layers beneath. Meibomian glands (located at the lid margins) are primarily responsible for lipid generation, and abnormal secretions from in these glands can lead to an unhealthy lipid layer in the tear film. The lipid secreted by the meibomian glands also retards evaporation from the preocular surface, lowers the surface tension of tears, prevents spillover of tears from the lid margin, prevents the contamination of the tear film by sebaceous lipids and prevents damage to the skin of the lid margin.

Abnormal meibomian gland secretions, a condition associated with obstruction and inflammation of the meibomian glands, is a widespread and chronic problem. Abnormal meibomian secretions is one of the most common causes of dry eye syndrome.

The signs and symptoms of dry eye are exacerbated by abnormalities in the lipid layer of the tear film, which is produced by the meibomian glands. Obstruction of the meibomian ducts causes accumulation of meibomian gland secretions, known as meibum. Accumulation of meibum within the meibomian gland can lead to inflammation of the gland and bacterial colonization. The colonizing bacteria have lipases that break the non-polar wax and sterol esters into triglycerides and free fatty acids (polar lipids), thus altering the normal composition of the meibum and the lipid layer of the tear film causing the tear film to become unstable, and the surface of the eye unwettable. Another potential mechanism of diseases associated with abnormal meibomian gland secretion is through quorum sensing, a means which by bacteria communicate with each other. When the normal bacterial flora balances change different genes in the bacteria are transcribed producing immune stimulating compounds. This leads to inflammation of the meibomian gland and altered secretions and gland obstruction.

Currently, there is no FDA approved treatment for disease characterized by abnormal meibomian gland secretions. As such, there exists a need for an ocular therapeutic for normalizing meibomian gland secretions which is comfortable upon administration to the eye, eye lid, eye lashes and/or eye lid margin or a subject, and at a safe dose particularly suitable for long term use. The present invention meets this need and other needs.

### SUMMARY OF THE INVENTION

The present invention is based, in part, upon the discovery that some non-aqueous components, (e,g, oils) are not appropriate for administering a pharmaceutically active agent to the meibomian gland orifice as they will cause irritation, destabilization of the tear film, blurring, and caking. The present invention is also based, in part, upon the discovery of ideal non-aqueous components suitable for the delivery of a pharmaceutically active agent to the meibomian gland orifice.

As such, the present invention provides novel topical ophthalmic formulations suitable for normalizing meibomian gland secretions which comprise a combination of ingredients capable of acting synergistically to normalize meibomian gland secretion (i.e., decrease meibomian secretion viscosity, increase secretions transparency to a colorless state and decrease the time (refractory period) between gland secretions) and relieve ocular discomfort, thereby treating and/or preventing at least one sign or symptom of dry eye (e.g. tear film break-up time, fluorescein staining, and/or ocular discomfort). in particular, the formulations described herein comprise a non-aqueous component and one or more active agents suitable for intermittent and/or repeated long term use for the treatment and/or prevention of abnormal meibomian secretions.

Also provided are methods for normalizing meibomian gland secretions by administering the topical ophthalmic formulations of the invention to a subject in need thereof. Such methods may comprise administering to a subject in need thereof the formulations of the invention an amount effective to decrease the meibomian secretion viscosity, increase secretions transparency to a colorless state, decrease the time (refractory period) between gland secretions) in the subject and/or reduce at least one sign or symptom of dry eye in the subject. Other methods of normalizing meibomian gland secretions provided herein may comprise administering to a subject in need thereof the formulations of the invention in an amount which is effective to normalize meibomian gland secretions in the subject. In one embodiment, the method for normalizing meibomian gland secretions comprises the steps of administering to a patient with abnormal meibomian gland secretions a non-aqueous formulation comprising an anti-infective, an anti-inflammatory, a hormonal agent, a neuronal agent, or any combination thereof.

Further provided are methods of treating or preventing dry eye disease, comprising administering the topical ophthalmic formulations of the invention, wherein the composition is administered in an amount effective to normalize meibomian gland secretions in the subject.

In any of the methods or formulations, the one or more active agents includes but is not limited to anti-infective agents, anti-inflammatory agents, neuronal agents, hormonal agents, or any combination thereof. The anti-infective agents, anti-inflammatory agents, neuronal agent and/or hormonal agents may be present or used in combination or alone. In a particular embodiment, the active agent is an anti-infective agent. Preferably, the anti-infective agent is a tetracycline antibiotic or a derivative or analog thereof. More preferably, the active agent is minocycline.

Examples of non-aqueous components suitable for use in the methods or formulations of the invention include but are not limited to oils such as castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof. In a particular embodiment, the non-aqueous component is castor oil and olive oil combination.

Preferably, the viscosity of the non-aqueous component is in a range which provides acute protection of the meibomian gland orifice, optimizes efficacy of supporting the tear film of the ocular surface, and increases the dwell time of the active agent to the meibomian gland orifice with minimal visual disruption (e.g., blurring), ocular irritation, and lid caking. Preferably, the viscosity of the non-aqueous component, ranges from about 50 centipoise (cps) to about 1000 cps, preferably about 50 cps to about 500 cps, more preferably about 50 cps to about 200 cps, even more preferably about 60 cps to about 120 cps.

In one embodiment of the invention, the active agent in the ophthalmic formulation of the invention is about 0.001% to about 5%, more preferably about 0.01% to about 3%, even more preferably about 0.01% to about 1% minocycline. In another embodiment the active agent in the ophthalmic formulation of the invention is about 0.001% to about 5%, more preferably about 0.01% to about 3%, even more preferably about 0.01% to about 1% of anti-infective agent, anti-inflammatory agent, neuronal agent, hormonal agent, or any combination thereof.

In one embodiment, the non-aqueous topical ophthalmic formulation of the invention comprises minocycline, light mineral oil, and mineral oil. In a particular embodiment, the non-aqueous topical formulation of the invention comprises 1.0% light mineral oil and 4.5% mineral oil and has a viscosity ranging from about 5 cps to about 10 cps. In another particular embodiment of the invention the minocycline is present at about 0.25% w/v.

In another embodiment, the non-aqueous topical ophthalmic formulation of the invention comprises minocycline, castor oil and olive oil, and has a viscosity ranging from about 80 cps to about 100 cps. In one particular embodiment the minocycline is present at about 0.25% w/v.

Also provided by the present invention are methods for delivery an active agent to the meibomian gland orifice of a subject comprising: (a) formulating the active agent in a non-aqueous solution having a viscosity ranging from about 50 centipoise (cps) to about 1000 cps, preferably about 50 cps to about 500 cps, more preferably about 50 cps to about 200 cps, even more preferably about 60 cps to about 120 cps; and (b) administering the formulation of step (a) to the eye lid, eye lashes, or eye lid margin of a subject using an applicator. The non-aqueous component provides acute protection of the meibomian gland orifice, optimizes efficacy of supporting the tear film of the ocular surface, and increases the dwell time of the active agent to the meibomian gland orifice thereby increasing therapeutic efficacy of the active agent, while the administration using an applicator increases the delivery of the active agent to the meibomian gland orifice.

Also featured are methods of evaluating the efficacy of and/or progress of normalizing meibomian gland secretions.

An additional feature is the use of a non-aqueous component and one or more active agents in the manufacture of a comfortable ophthalmic formulation for administration to the eye lid, eye lashes and eye lid margin, wherein the non-aqueous component has a viscosity ranging from about 50 centipoise (cps) to about 1000 cps, preferably about 50 cps to about 500 cps, more preferably about 50 cps to about 200 cps, even more preferably about 60 cps to about 120 cps, and the one or more active agents includes but is not limited to anti-infective agents, anti-inflammatory agents, neuronal agents, hormonal agents, or any combination thereof.

Also featured are methods of evaluating the efficacy of an ophthalmic formulation for the treatment of abnormal meibomian gland secretions. Such methods comprise a) evaluating and recording baseline measurements of tear film break-up time (TFBUT) and ocular protection index (e.g., by performing slit lamp evaluation, fluorescein staining), and establishing a baseline measurement of meibomian gland secretions quality (e.g., by evaluating one or more of secretion viscosity, secretion color, gland alignment, vascularity pattern, vascularity redness, hyperkeratinization, posterior lid edge, lash, mucocutaneous junction, perigland redness, gland geometry and gland height) in the eye(s) of a subject; b) administering an ophthalmic formulation (e.g., an ophthalmic formulation comprising an active agent such as anti-infective agent, an anti-inflammatory agent, a neuronal agent, a hormonal agent or any combination thereof, and a non-aqueous component having a viscosity ranging from 60-120 cps) to the eye lid, eye lashes, or eye lid margin of the subject; and c) re-evaluating and recording TFBUT and OPI in the eye(s) of the subject (e.g., by at least one of slit lamp evaluation, fluorescein imaging) and evaluating meibomian gland secretion quality (e.g., by evaluating one or more of secretion viscosity, secretion color, gland alignment, vascularity pattern, vascularity redness, hyperkeratinization, posterior lid edge, lash, mucocutaneous junction, perigland redness, gland geometry and gland height); wherein the comparative results of each evaluation in (a) and (c) indicates whether the ophthalmic formulation is efficacious in treating or preventing abnormal meibomian gland secretions in the subject.

Further featured are kits for the shipping, storage or use of the formulations, as well the practice of the methods.

Other features and advantages of the invention will become apparent from the following detailed description and claims.

### BRIEF DESCRIPTION OF THE DRAWINGS

FIG. 1 is a graph depicting the percent of total of meibomian gland marked by Lissamine green at various timepoints (1, 2, 5, 10, 20 and 30 minutes) after administration to the eye of participating subjects (N=6) using a low viscosity (i.e., 5-10 cps), mid viscosity (i.e., 80-100 cps), and high viscosity (i.e., >10,000 cps) oil formulation.

FIG. 2 is a graph depicting the mean comfort of a low viscosity (i.e., 5-10 cps), mid viscosity (i.e., 80-100 cps), and high viscosity (i.e., >10,000 cps) oil formulation at various timepoints (1, 2, 5, 10, 20 and 30 minutes) after administration to the eye of participating subjects (N=6).

FIG. 3 is a graph depicting the mean percent blurring present in the eyes of participating subjects (N=6) at various timepoints (1, 2, 5, 10, 20 and 30 minutes) after administration of a low viscosity (i.e., 5-10 cps), mid viscosity (i. e., 80-100 cps), and high viscosity (i.e., >10,000 cps) oil formulation into the eyes of the subjects.

### DETAILED DESCRIPTION

For convenience, before further description of the present invention, certain terms employed in the specification, examples, and appended claims are collected here. These definitions should be read in light of the remainder of the disclosure and understood as by a person of skill in the art.

The term “abnormal meibomian gland secretion” refers to a meibomian gland secretion with increased viscosity, opacity, color and/or an increased time (refractory period) between gland secretions.

The term “aqueous” typically denotes an aqueous composition wherein the carrier is to an extent of >50% , more preferably >75% and in particular >90% by weight water.

The term “blepharitis” refers to a disorder comprising inflammation of the lid margin in which abnormal meibomian gland secretions plays a role and lid keratinization, lid margin rounding, obscuration of the grey line, increased lid margin transparency, and increased vascularity are observed. Although the terms meibomian gland dysfunction and meibomianitis are commonly referred to as blepharitis by most investigators, it is important to note that these arc distinct diseases associated with abnormal meibomian gland secretions and that the terms are not interchangeable.

The term “comfortable” as used herein refers to a sensation of physical well being or relief, in contrast to the physical sensation of pain, burning, stinging, itching, irritation, or other symptoms associated with physical discomfort.

The term “comfortable ophthalmic formulation” as used herein refers to an ophthalmic formulation which provides physical relief from signs or symptoms associated with lid margin inflammation and/or ocular discomfort, and only causes an acceptable level of pain, burning, stinging, itching, irritation, or other symptoms associated with ocular discomfort, when instilled in the eye.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of lid margin irritation, or prevent or treat lid margin inflammation. The effective amount may vary depending on such factors as the disease or condition being treated, the particular composition being administered, or the severity of the disease or condition. One of skill in the art may empirically determine the effective amount of a particular agent without necessitating undue experimentation.

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid (aqueous or non-aqueous) or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils such as castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof, (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “preventing,” when used in relation to a condition, such as abnormal meibomian gland secretions, is art-recognized, and refers to administration of a composition which reduces the frequency of, or delays the onset of, signs and/or symptoms of a medical condition in a subject relative to a subject which does not receive the composition.

The term “treating” is an art-recognized term which refers to curing as well as ameliorating at least one symptom of any condition or disease.

1. Pharmaceutical Compositions

The invention features novel topical pharmaceutical compositions comprising an effective amount of an active agent, including but not limited to an anti-infective agent, an anti-inflammatory agent, a neuronal agent, a hormonal agent, or any combination thereof, and a non-aqueous component (e.g., a pharmaceutically acceptable non-aqueous carrier) having a viscosity ranging from about 50 cps to about 1000 cps, preferably about 50 cps to about 500 cps, more preferably about 50 cps to about 200 cps, even more preferably about 60 cps to about 120 cps, useful for the treatment and prevention of abnormal meibomian gland secretions. The active agents may be present in combination or alone. An effective amount of the formulations of the invention may be used to normalize meibomian gland secretions, thereby treating diseases associated therewith (e.g., dry eye). Signs and symptoms of abnormal meibomian secretions include but are not limited to increased meibomian secretion viscosity, opacity, color, as well as an increase in the time (refractory period) between gland secretions. Signs and symptoms of diseases associated with abnormal meibomian gland secretions include but are not limited to dry eye, redness of the eyes, itching and/or irritation of the eyelid margins and edema, foreign body sensation, and matting of the lashes. Such formulations provide a comfortable ophthalmic formulation when instilled in the eye and have enhanced efficacy and duration of action over formulations of active agents (e.g., anti-infective agents) that are not combined with such non-aqueous components as described herein.

The extraordinary efficacy of these formulations is attributed to, among other things, the synergistic effect of the combination of ingredients in them. The active agent component improves treats, relieves, inhibits, prevents, or otherwise decreases the signs and symptoms of dry eye by normalizing meibomian gland secretions (e.g., decreasing meibomian secretion viscosity, increasing secretions transparency to a colorless state and decreasing the time (refractory period) between gland secretions in the subject). The non-aqueous component having a viscosity ranging from about 50 cps to about 1000 cps, preferably about 50 cps to about 500 cps, more preferably about 50 cps to about 200 cps, even more preferably about 60 cps to about 120 cps provides ocular surface protection via enhancement of the tear film (as evident by increased tear film break up time (TFBUT) and/or the ocular protection index (OPI)), acute protection of the meibomian gland orifice, and increases the therapeutic efficacy of the active agent component by extending the residence time of the active agent to the meibomian gland orifice, with minimal visual disruption (e.g., blurring), ocular irritation, or lid caking. As such, the compositions of the invention are comfortable upon application to the eye lid, eye lashes, or eye lid margin of a subject, and may be used for relief of acute or chronic abnormal meibomian gland secretions, and are particularly suitable for both intermittent and long term use.

In certain embodiments, the compositions may treat or prevent abnormal meibomian gland secretions by normalizing meibomian gland function. (i.e., decreasing the meibomian secretion viscosity, increasing secretions transparency to a colorless state and decreasing the time (refractory period) between gland secretions).

Anti-inflammatory agents may be steroids or non-steroidal anti-inflammatory agents. Exemplary steroids include, but are not limited to, hydrocortisone, prednisolone, dexamethasone, and fluorometholone. Exemplary non-steroidal anti-inflammatory agents include, but are not limited to, ketorolac tromethamine (Acular®) (and the other compounds described as being ophthalmologically effective in U.S. Pat. No. 4,454,151 to Waterbury, issued Jun. 12, 1984, the pertinent portions of which are incorporated herein by reference), indomethacin, flurbiprofen sodium, nepafenac, bromfenac, suprofen and diclofenac (Voltaren®) (and the other compounds described as being ophthalmologically effective in U.S. Pat. No. 4,960,799 to Nagy, issued Oct. 2, 1990, the pertinent portions of which are incorporated herein by reference), including the esters and pharmaceutically acceptable salts thereof.

Exemplary anti-infective agents include, but are not limited to, mupirocin; antianaerobic anti-infectives, such as chloramphenicol and clindamycin, antifungal antibiotic anti-infectives, such as amphotericin b, clotrimazole, fluconazole, and ketoconazole; macrolide antibiotic anti-infectives, such as azi-thromycin and erythromycin; miscellaneous P-lactam antibiotic anti-infectives, such as aztreonam and imipenem; penicillin antibiotic anti-infectives, such as nafcillin, oxacillin, penicillin G, and penicillin V; quinolone antibiotic anti-infectives, such as ciprofloxacin and norfloxacin; tetracycline antibiotic anti-infectives, such as doxycycline, minocycline, and tetracycline; and antiprotozoal anti-infectives, such as atovaquone and dapsone.

Exemplary anti-inflammatory agent/anti-infective agent combinations include, but are not limited to, combinations of any of the above-described anti-inflammatory and anti-infective agents. Other exemplary commercially available combinations are Tobradex® (1 mg dexamethasone and 3 mg tobramycin per g), Zylet® (loteprednol etabonate 0.5% and tobramycin 0.3% ophthalmic suspension) and Pred G® (gentamicin sulfate equivalent to 0.3% gentamicin base; prednisolone acetate (microfine suspension) 1.0%).

Exemplary hormonal agents include, but are not limited, any molecule that is produced by a specific cell or tissue and causes a change or activity in a cell or tissue located elsewhere in an organism. Amine-derived hormones are derivatives of the amino acids tyrosine and tryptophan. Examples, include, but are not limited to, catecholamines and thyroxine. Peptide hormones comprise chains of amino acids. Examples of small peptide hormones include, but are not limited to, TRH and vasopressin. Examples of protein hormones include, but are not limited to, insulin and growth hormone. More complex protein hormones bear carbohydrate side chains and are called glycoprotein hormones. Luteinizing hormone, follicle-stimulating hormone and thyroid-stimulating hormone are exemplary glycoprotein hormones. Lipid and phospholipid-derived hormones derive from lipids such as linoleic acid and arachidonic acid and phospholipids. The main classes are the steroid hormones that derive from cholesterol and the eicosanoids. Examples of steroid hormones include, but are not limited to, testosterone and cortisol. Sterol hormones include, but are not limited to, calcitriol. Examples of eicosanoids include the widely studied pro staglandins.

Exemplary neuronal agents include, but are not limited to, neurotransmitters and neuropeptides. The term “neurotransmitter” as used herein means any molecule or compound, which is released from the axon of one neuron and binds to a specific site in the dendrite of an adjacent neuron, thus triggering a nerve impulse. A neurotransmitter may be, for example, a small molecule, a peptide, an amino acid, a hormone, a protein, a vitamin, or a free radical. The term “neuropeptide” as used herein means a peptide with a direct synaptic effect (i.e., a peptide that is a neurotransmitter) and/or an indirect effect on synaptic transmission. Neuropeptides may be released from neurons or from non-neuronal cells, and may also act as hormones.

Exemplary neurotransmitters include, but are not limited to, acetylcholine, ATP, glycine, glutamate, dopamine, norepinephrine, epinephrine, octopamine, serotonin (5-hydroxytryptamine), beta-alanine, histamine, gamma-aminobutyric acid (GABA), taurine, aspartate and nitric oxide. Accordingly, neurotransmitters may be small molecules, peptides, amino acids, hormones, proteins, vitamins or free radicals. In other embodiments, the composition comprises at least one neuropeptide. In addition to “classic” neurotransmitters such as those listed above, there is a growing list of peptide molecules produced and released in the nervous system that act as neurotransmitters or which influence synaptic transmission. These neurotransmitters are also known in the art as “neuro secretory substances.” Exemplary neuropeptides include, but are not limited to, hypothalamic hormones such as oxytocin (9 amino acid residues, “a.a.r.”) and vasopressin (9 a.a.r.); hypothalamic releasing and inhibiting hormones such as corticotropin releasing hormone (CRH) (41 a.a.r.), growth hormone releasing hormone (GHRH) (44 a.a.r.), luteinizing hormone releasing hormone (LHRH) (10 a.a.r.), somatostatin growth hormone release inhibiting hormone (14 a.a.r. plus several forms) and thyrotropin releasing hormone (TRH) (3 a.a.r.); tachykinins such as neurokinin a (substance K) (10 a.a.r.), neurokinin b (10 a.a.r.), neuropeptide K (36 a.a.r.) and substance P (11 a.a.r.); opioid peptides such as b-endorphin (30 a.a.r.), dynorphin (17 a.a.r. and other forms) and met- and leu-enkephalin (5 a.a.r.); NPY and related peptides such as neuropeptide tyrosine (NPY) (36 a.a.r.), pancreatic polypeptide (36 a.a.r) and peptide tyrosine-tyrosine (PYY) (36 a.a.r.); VIP-glucagon family members such as glucogen-like peptide-1 (GLP-1) (29 a.a.r.), peptide histidine isoleucine (PHI) (27 a.a.r.), pituitary adenylate cyclase activating peptide (PACAP) (27 or 38 a.a.r.) and vasoactive intestinal polypeptide (VIP) (28 a.a.r.); as well as many other peptides such as brain natriuretic peptide (32 a.a.r.), calcitonin gene-related peptide (CGRP) (a- and b-form) (37 a.a.r.), cholecystokinin (CCK) (8 a.a.r. and other forms), galanin (29 or 30 a.a.r.), islet amyloid polypeptide (IAPP) or amylin (37 a.a.r). melanin concentrating hormone (MCH) (19 a.a.r.), melanocortins (ACTH, a-MSH and others), neuropeptide FF (F8Fa) (8 a.a.r), neurotensin (13 a.a.r.), parathyroid hormone related protein (34 or 37 a.a.r.), Agouti gene-related protein (AGRP) (131 a.a.r.), cocaine and amphetamine regulated transcript (CART)/peptide, endomnorphin-1 and -2 (both 4 a.a.r.), 5-HT-moduline (4 a.a.r.), hypocretins/orexins (29 or 39 a.a.r.), nociceptin/orphanin FQ (17 a.a.r.), nocistatin (17 a.a.r.), prolactin releasing peptide (20 or 31 a.a.r.), secretoneurin (33 a.a.r.) and urocortin (40 a.a.r.; 45% sequence identity with CRH).

Pharmaceutical ophthalmic formulations typically contain an effective amount, e.g., about 0.001% to about 10% wt/vol., preferably about 0.001% to about 5%, more preferably about 0.01% to about 3%, even more preferably about 0.01% to about 1% of an active agent ingredient (e.g., an anti-infective agent and/or an anti-inflammatory agent) suitable for short or long term use for normalizing meibomian gland secretions. The amount of active ingredient will vary with the particular formulation and the disease state for which it is intended. For example., where the active ingredient is an anti-infective and/or anti-inflammatory agent, e.g., a tetracycline derivative, an effect amount ranges from about 0.001% to about 5%, more preferably about 0.01% to about 3%, even more preferably about 0.01% to about 1%.

Preferably, the effective amount of active agent present in the formulations should be sufficient to treat or prevent abnormal meibomian gland secretions. In certain embodiments, the active agent may treat or prevent abnormal meibomian gland secretions by normalizing (e,g, decreasing the meibomian secretion viscosity, increasing secretions transparency to a colorless state and decreasing the time (refractory period) between gland secretions) meibomian gland secretions.

The pharmaceutical compositions of the invention described above may additionally comprise other active ingredients, including, but not limited to, and vasoconstrictors, antiallergenic agents, anesthetics, analgesics, dry eye agents (e.g. secretagogues, mucomimetics, polymers, lipids, antioxidants), etc., or be administered in conjunction (simultaneously or sequentially) with pharmaceutical compositions comprising other active ingredients, including, but not limited to, and vasoconstrictors, antiallergenic agents, anesthetics, analgesics, dry eye agents (e.g. secretagogues, mucomimetics, polymers, lipids, antioxidants), etc.

The active agents of the pharmaceutical compositions may be in the form of a pharmaceutically acceptable salt.

Preferably, the pharmaceutical compositions according to the present invention will be formulated as solutions, suspensions and other dosage forms for topical administration to the eye lid, eye lashes and eye lid margin in order to deliver the formulation to the meibomian gland orifice. Liquid (aqueous or non-aqeuous) solutions are generally preferred, based on ease of formulation, as well as a patient's ability to easily administer such compositions by means of applying the formulation to the eye lid, eye lashes and eye lid margin. Application may be performed with an applicator, such as the patient's finger, a Wek-Cel, Q-tip, or other device capable of delivering the formulation to the eye lid, eye lashes or eye lid margin in order to deliver the formulation to the meibomian gland orifice. However, the compositions may also be ointments, suspensions, viscous or semi-viscous gels, or other types of solid or semi-solid compositions.

Any of a variety of carriers may be used in the formulations of the present invention. In a preferred embodiment, the carrier is a non-aqueous carrier (e.g., oil, or oil mixture) having a viscosity in a range which provides ocular surface protection via enhancement of the anti-evaporative lipid layer of the tear film (as evident by increased tear film break up time (TFBUT) and/or the ocular protection index (OPI)), acute protection of the meibomian gland orifice, and increases the therapeutic efficacy of the active agent component by extending the residence time of the active agent to the meibomian gland orifice, with minimal visual disruption (e.g., blurring), ocular irritation, or lid caking. Preferably, the viscosity of the non-aqueous carrier ranges from about 50 cps to about 1000 cps, preferably about 50 cps to about 500 cps, more preferably about 50 cps to about 200 cps, even more preferably about 60 cps to about 120 cps. In certain embodiments, the non-aqueous carrier comprises an oil, e.g., castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof.

In certain embodiments, a tear substitute acts as the pharmaceutical carrier. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; polymers, such as polyvinyl alcohol, polyvinylpyrrolidone, and povidone; carbomers, such as carbomer 934P, carbomer 941, carbomer 940 and carbomer 974P; and gums such as HP-guar. Many such tear substitutes are commercially available, which include, but are not limited to cellulose esters such as Bion Tears®, Celluvisco, Genteal®, OccuCoat®, Refresh®, Teargen II®, Tears Naturale®, Tears Naturale II®, Tears Naturale Free®, and TheraTears®; and polyvinyl alcohols such as Akwa Tears®, HypoTears®, Moisture Eyes®, Murine Lubricating®, Systane® Lubricant Eye Drops, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

In certain embodiments, the tear substitute contains hydroxypropylmethylcellulose. In certain embodiments, the tear substitute is Genteal® lubricating eye drops. GenTeal® (CibaVision-Novartis) is a sterile lubricant eye drop containing hydroxypropyl methylcellulose 3 mg/g and preserved with sodium perborate.

In another embodiment, the tear substitute comprises carboxymethyl cellulose sodium. For example, without limitation, the tear substitute which comprises carboxymethyl cellulose sodium is Refresh® Tears. Refresh® Tears is a lubricating formulation similar to normal tears, containing a, mild non-sensitizing preservative, stabilised oxychloro complex (Purite™), that ultimately changes into components of natural tears when used.

Additional carriers may optionally be included in the formulations of the present invention. Examples of additional carriers include for example, water, mixtures of water and water-miscible solvents, such as C₁- to C₇-alkanols, vegetable oils, mineral oils or other oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenyhnercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary-contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to adjust (e.g., increase) the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

In embodiments wherein the formulation is an ointment, a preferred ointment base used to prepare the ophthalmic ointment of the present invention may be one that has been used in conventional ophthalmic ointments. In particular, the base may be liquid paraffin, white petrolatum, purified lanolin, gelation hydrocarbon, polyethylene glycol, hydrophilic ointment base, white ointment base, absorptive ointment base, Macrogol (Trade Name) ointment base, simple ointment base, and the like.

The ophthalmic ointment may comprise further conventional excipients other than the ointment base in the range of without affecting the intended functions and stability of vancomycin hydrochloride to be contained. Examples of such excipients include antiseptics such as parahydroxybenzoate, chlorobutanol, benzalkonium chloride and the like; surfactants such as polysorbate 80, polyoxyl 40 stearate, polyoxyethylene hydrogenated castor oil and the like; stabilizers such as sodium edetate, citric acid, and salts thereof; alcohols such as glycerol, lanolin alcohol, cetanol and the like; esters such as isopropyl myristate, ethyl linoleate and the like; and oils such as olive oil and triglycerides of middle-chained fatty acids.

2. Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

Ophthalmic ointments may be produced as follows: if necessary, antiseptics, surfactants, stabilizers, alcohols, esters or oils are blended with an ointment base such as liquid paraffin or white petrolatum placed in a mortar or a mixing machine for ointment to form a mixture. This is followed by addition of vancomycin hydrochloride, and the resulting mixture is mixed until uniform and kneaded to form the ophthalmic ointment. The ointment thus prepared is filled into a bottle or tube for ointment to obtain the ophthalmic ointment containing vancomycin hydrochloride of the present invention.

3. Methods of Use

The invention features methods of treating or preventing abnormal meibomian gland secretions in a subject comprising use of the novel formulations described above. For example, a method of treating or preventing abnormal meibomian gland secretions may comprise administering to the eye lid, eye lashes, or eye lid margin of a subject in need thereof a formulation comprising an effective amount of an active agent including but not limited to an anti-infective agent, an anti-inflammatory agent, a neuronal agent a hormonal agent, or any combination thereof, and a non-aqueous component (e.g., a pharmaceutically acceptable non-aqueous carrier) having a viscosity ranging from about 50 cps to about 1000 cps, preferably about 50 cps to about 500 cps, more preferably about 50 cps to about 200 cps, even more preferably about 60 cps to about 120 cps. The active agents may be administered in combination or alone. In embodiments wherein the agents are administered in combination, they may be administered simultaneously or sequentially. Preferably, the non-aqueous component is an oil, e.g., castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof. For example, the non-aqueous component is castor oil and/or olive oil, alone or in combination. Such administration may reduce at least one sign of abnormal meibomian gland secretions in the subject and may operate by normalizing meibomian gland secretions in the subject.

The effective amount of active agent to include in a given formulation, and the efficacy of a formulation for normalizing meibomian gland secretions, may be assessed by one or more of the following: slit lamp evaluation, fluorescein staining, tear film breakup time, and evaluating meibomian gland secretions quality (by evaluating one or more of secretion viscosity, secretion color, gland alignment, vascularity pattern, vascularity redness, hyperkeratinization, posterior lid edge, lash, mucocutaneous junction, perigland redness, gland geometry and gland height).

The effective amount of active agent(s) in the formulation will depend on absorption, inactivation, and excretion rates of the drug as well as the delivery rate of the active agent(s) from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the composition and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The combined use of several active agents formulated into the compositions of the present invention may reduce the required dosage for any individual component because the onset and duration of effect of the different components may be complimentary. In such combined therapy, the different active agents may be delivered together or separately, and simultaneously or at different times within the day.

4. Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, ointments suspensions or formulations, tablets, or capsules of this invention. The kits can be designed to facilitate one or more aspects of shipping, use, and storage.

The kits may optionally include instructional materials containing directions (i.e., protocols) disclosing means of use of the formulations provided therein. The kits may also optionally include a topical applicator to facilitate administration of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

The invention now being generally described, it will be more readily understood by reference to the following examples which are included merely for purposes of illustration of certain aspects and embodiments of the present invention, and are not intended to limit the invention in any way.

### Example 1

### Safety and Efficacy of Tobradex® in Treating Blepharitis

It is hypothesized that topical combination anti-infective/anti-inflammatory treatments will reduce symptoms associated with abnormal meibmoian gland secretions, for example, in a subject suffering from blepharitis.

A randomized double-masked, placebo controlled, study of the safety and efficacy of Tobradex®, an anti-infective/anti-inflammatory combination therapeutic, in subjects with blepharitis is conducted as follows. Lid margin health is evaluated at Visit 1. The meibomian glands, lower eyelid only, of all qualified subjects is photographed, numbered and graded at Visit 1. The subjects then receive one of two treatments BID for 28 days. At Visit 2 (Day 14) and Visit 3 (Day 28), the subjects undergo the same lid margin and meibomian gland evaluations that were conducted at Visit 1.

Qualified subjects are randomized to receive Tobradex® ointment (1 mg dexamethasone and 3 mg tobramycin per g) or Refresh PM Ointment (placebo) bilaterally BID for 28 days. All subjects apply a 1.5 cm ribbon of medication to the lid margin with a finger tip. The efficacy of the treatments are primarily assessed on each of the three visits by evaluating lid vascularity redness and ocular discomfort, and secondarily assessed by evaluating lid vascularity, secretion quality, orifice geometry, perigland redness, hyperkeratinization, tear film break up time (TFBUT), tear film break up pattern (TFBUP), corneal and conjunctival staining, alignment, cross-sectional posterior lid margin, lash, mucocutaneous junction, conjunctival redness and corneal and lid sensitivity. In particular, the procedures performed at each study visit are as follows:

Visit 1 (Day 0): informed consent, demographic data, medical/medication history, ocular discomfort, symptom evaluation, urine pregnancy test (if applicable), blink rate, visual acuity, slit lamp biomicroscopy, conjunctival redness, tear meniscus height, lid margin evaluation, meibomian gland evaluation, TFBUT, fluorescein staining, lid margin sensitivity, corneal sensitivity, intraocular pressure, dilated fundus examination, randomize qualified subjects to receive study medication BID, instruct/dispense medication and diary.

Visit 2 (Day 14): medical/medication history update, ocular discomfort, symptom evaluation, blink rate, visual acuity, slit lamp biomicroscopy, conjunctival redness, tear meniscus height, lid margin evaluation, meibomian gland evaluation, TFBUT, fluorescein staining, lid margin sensitivity, corneal sensitivity, intraocular pressure, collect/dispense study medication and diary.

Visit 3 (Day 28): medical/medication history update, collect study medication, ocular discomfort, symptom evaluation, urine pregnancy test (if applicable), blink rate, visual acuity, slit lamp biomicroscopy, conjunctival redness, tear meniscus height, lid margin evaluation, TFBUT, fluorescein staining, lid margin sensitivity, corneal sensitivity, intraocular pressure, dilated fundus examination and exit subject from study. Adverse events (both elicited and observed) will be monitored throughout the study and promptly reviewed and documented.

### Example 2

### Delivery of Lissamine Green to Meibomian Glands via Non-Aqueous Solutions

The following study describes the identification of the ideal viscosity of non-aqeuous formulations for delivery of an active agent to meibomian glands of the eyelid. Lissamine green was used as a delivery marker.

Three non-aqueous formulations comprising minocycline were prepared covering a range of oil concentrations and viscosities:

### Low Viscosity/Low Oil Concentration Preparation.

A non-aqueous solution of minocycline (2.5 mg/mL; 0.25%) was prepared in an oil in water emulsion by dissolving 25 mg of minocycline hydrochloride in 10 mL of the emulsion (Bausch & Lomb SOOTH). The emulsion consisted of 1.0% light mineral oil and 4.5% mineral oil and two surfactants. The approximate viscosity of this formulation was 5-10 centipoise (cps).

### Medium Viscosity/High Oil Concentration Preparation.

A non-aqueous formulation of minocycline (2.5 mg/mL; 0.25%) was prepared in an oil formulation by suspending 25 mg of minocycline hydrochloride in 10 mL of a light viscosity oil mixture (Art of Shaving Pre-Shave Oil) The oil mixture consisted of castor oil, olive oil, and essential oil. The approximate viscosity of this formulation was 80-100 cps.

### High Viscosity/High Oil Concentration Preparation.

A non-aqueous formulation of minocycline (2.5 mg/mL; 0.25%) was prepared in an non-aqueous oil based formulation by suspending 25 mg of minocycline hydrochloride in 10 mL ointment base (GenTeal PM Ointment, Novartis Ophthalmics). The ointment base consisted of mineral oil 15% and white petrolatum 85% . The approximate viscosity of this formulation was >10,000 cps.

Baseline ocular exams were performed by an ophthalmologist on subjects to identify the number of meibomian glands present in the right (OD) and left (OS) eyelids of each subject. A total of 4 male subjects were evaluated.

The low, medium and high viscosity non-aqueous minocycline formulations described above were mixed with 2% of Lissamine green. One drop of the low, medium or high viscosity formulation was instilled in the right eye (OD) of each subject, and one drop of the low, medium, or high formulation was instilled in the left eye (OS) of each subject. The formulations were placed on the lid margin to provide optimal application to meibomian gland orifices.

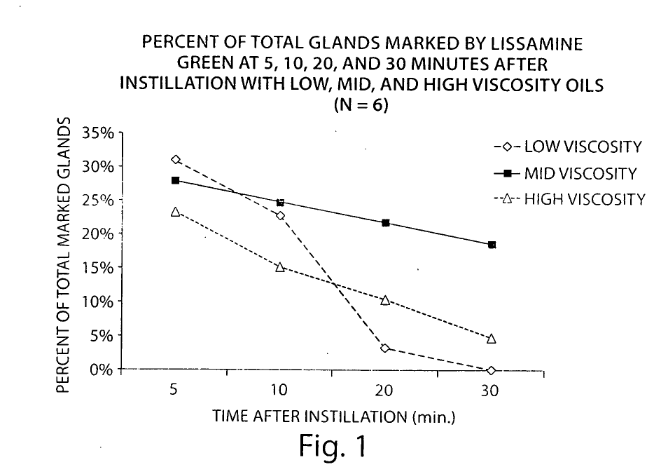

Subjects were asked to subjectively grade blurring (present or absent) and comfort of the formulation in each eye based on a comfort scale of 0 to 10 (0 indicating most comfortable, 10 indicating least comfortable) immediately (0), 1, 2, 5, 10, 20 and 30 minutes after administration of the non-aqueous solutions. Additionally, the number of glands marked with Lissamine green in the right and left eyelids of each subject were counted and recorded 5, 10, 20, and 30 minutes after administration of the non-aqueous formulations. The results are shown in FIGS. 1-3 and summarized below.

As shown in FIG. 1, the percent of total meibomian glands marked by Lissamine green over a 30 minute period was highest for the mid viscosity oil/minocycline formulation (80-100 cps). The percent of total marked glands over a 30 minute period was lowest for the low viscosity oil/minocycline formulation (5-10 cps).

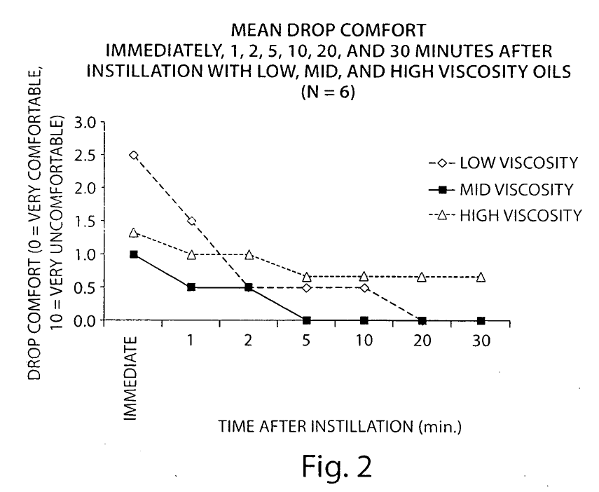

As shown in FIG. 2, the mean drop comfort over a 30 minute period was most comfortable for the mid viscosity oil/minocycline formulation (80-100 cps). The mean drop comfort over a 30 minute period was least comfortable for the high viscosity oil/minocycline formulation (>10,000 cps).

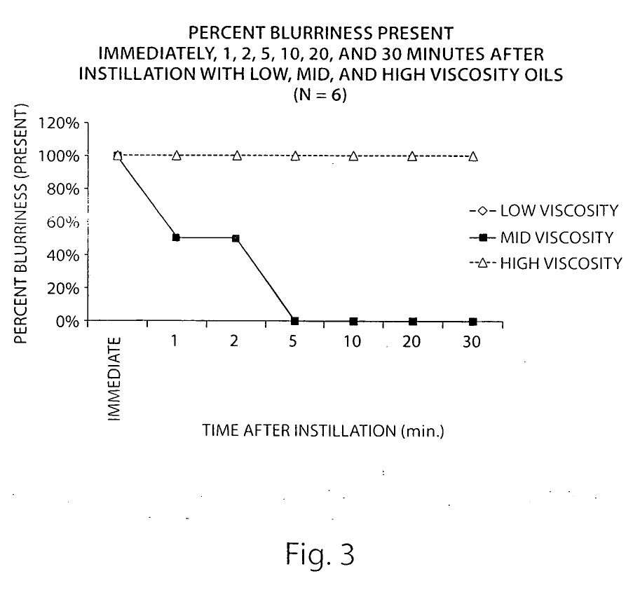

As shown in FIG. 3, the mean blurriness over a 30 minute period was least blurring for the mid and low viscosity oil/minocycline formulations (80-100 cps and 5-10 cps, respectively). The mid and low viscosity oil/minocycline formulations had the same percent of blurring present over the 30 minute period. Because the results for the mid and low viscosity formulations were identical, the line representing the low viscosity/minocycline formulation is not visible in the graph shown in FIG. 3. The mean blurriness over a 30 minute period was most blurring for the high viscosity oil/minocycline formulation (i.e., >10,000 cps).

In summary, the mid viscosity oil/minocycline formulation was the most effective for delivering an active medication to the meibomian glands, was the most comfortable, and caused the least blurring (although comparable to low viscosity oil) over a 30 minute period. As demonstrated in this pilot study, not all oils are created equal when used to deliver medication to the meibomian glands. A mid viscosity oil (i.e., 80-100 cps) is a more effective carrier than a low viscosity oil (i.e., 5-10 cps) or high viscosity oil (i.e., >10,000 cps).

### Equivalents

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.
Sullivan, D. A. et al. (2000) Invest. Ophthalmol. Vis. Sci. 41(12):3732-3742.
Mathers, W. D. Meibomian Gland Disease. In: Pflugelder, S. et al, editors. Dry Eye and Ocular Surface Disorders. Marcel Dekker, Inc. New York.
Bron, A. J., et al. (1991) Eye 5:395-411.
Cassin, et al. Dictionary of Eye Terminology. 4^{th} Ed. Gainesville, Fla. Triad Communications, Inc. (2001).

What is claimed is:

1. A pharmaceutical composition formulated for topical ophthalmic use comprising a combination of:
(a) one or more active agents selected from the group consisting of an anti-infective agent, an anti-inflammatory agent, a neuronal agent, a hormonal agent, and any combination thereof; and
(b) a non-aqueous component having a viscosity ranging from about 60-120 centipoise.

2. The composition of claim 1, wherein the non-aqueous component is an oil selected from the group consisting of castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof.

3. The composition of claim 1, wherein the one or more active agents is at least an anti-infective agent.

4. The composition of claim 3, wherein the anti-infective agent is a tetracycline antibiotic or a derivative thereof.

5. The composition of claim 4, wherein the tetracycline antibiotic is minocycline.

6. A non-aqueous topical ophthalmic formulation comprising an oil selected from the group consisting of castor oil, olive oil, and a combination thereof, and minocycline.

7. A method of treating dry eye disease, comprising administering to a subject in need thereof a pharmaceutical composition comprising a combination of:
(a) one or more active agents selected from the group consisting of an anti-infective agent, an anti-inflammatory agent, a neuronal agent, a hormonal agent, and any combination thereof; and
(b) a non-aqueous component having a viscosity ranging from about 60-120 centipoise;
in an amount effective to normalize meibomian gland secretions in the subject.

8. A method of normalizing meibomian gland secretions, comprising administering to a subject in need thereof a pharmaceutical composition comprising:
(a) one or more active agents selected from the group consisting of an anti-infective agent, an anti-inflammatory agent, a neuronal agent, a hormonal agent, and any combination thereof; and
(b) a non-aqueous component having a viscosity ranging from about 60-120 centipoise; in an amount effective to decrease the meibomian secretion viscosity, increase secretion transparency to a colorless state and decrease the time (refractory period) between gland secretions) in the subject.

9. A method for increasing the therapeutic efficacy of an active agent to the meibomian gland orifice of a subject comprising:
(a) formulating the active agent in a non-aqueous solution having a viscosity ranging from about 60-120 centipoise;
(b) administering the formulation of step (a) to the eye lid, eye lashes, or eye lid margin of a subject using an applicator.

10. A method for delivering an active agent to the meibomian gland orifice of a subject comprising:
(a) formulating the active agent in a non-aqueous solution having a viscosity ranging from about 60-120 centipoise;
(b) administering the formulation of step (a) to the eye lid, eye lashes, or eye lid margin of a subject using an applicator.
